(19) United States
(12) Patent Application Publication
(10) Pub. No.: US 2018/0180635 A1
(43) Pub. Date: Jun. 28, 2018

(54) AUTOMATIC DIAGNOSTIC LABORATORY AND LABORATORY INFORMATION MANAGEMENT SYSTEM FOR HIGH THROUGHPUT

(71) Applicant: Counsyl, Inc., South San Francisco, CA (US)

(72) Inventors: Kyle Allen LAPHAM, San Francisco, CA (US); A. Scott PATTERSON, South San Francisco, CA (US); Kevin R. HAAS, Berkeley, CA (US); Christopher WONG, South San Francisco, CA (US); Taj MORTON, South San Francisco, CA (US); Ethan NASH, South San Francisco, CA (US); Jonas NEUBERT, South San Francisco, CA (US)

(21) Appl. No.: 15/852,516

(22) Filed: Dec. 22, 2017

Related U.S. Application Data
(60) Provisional application No. 62/438,262, filed on Dec. 22, 2016.

(51) Int. Cl. G01N 35/00; G16H 40/60; B25J 9/16

(52) U.S. Cl. CPC G01N 35/00871; G01N 2035/0091; B25J 9/1682; G16H 40/60

(57) ABSTRACT

A system for managing information in a laboratory is disclosed. The system can receive sample information, the sample information including at least one sample identifier and sample order information. The system can send movement information to one or more robotics units based on at least the sample identifier. The system can perform, on at least one identified sample, one or more analytical functions to generate results data. The system can organize the results data based on the sample order information.

### CROSS-REFERENCE TO RELATED APPLICATIONS

This application claims priority benefit of U.S. Provisional Patent Application No. 62/438,262 filed Dec. 22, 2016, the entire content of which is hereby incorporated by reference herein for all purposes.

### FIELD OF THE INVENTION

The invention relates generally to an automatic diagnostic laboratory for high throughput and management of processes in a laboratory environment.

### BACKGROUND OF THE INVENTION

A laboratory information management system (LIMS), also referred to as a laboratory management system (LMS) or a laboratory information system (LIS), is a system for modernizing functions within a laboratory that have traditionally been performed manually or semi-manually. A LIMS system may include but is not limited to a server or host computer, database, management software, and may be coupled to associated laboratory instrumentation for performing respective laboratory functions. A LIMS system will generally assist laboratory personnel in tracking, analyzing, sorting, and routing laboratory samples throughout complex laboratory processes in an efficient and cost-effective manner

Advantages of LIMS systems include, but are not limited to, enhanced sample management, quality control, chain of custody, and report generation. A LIMS system also permits flexible control of access to laboratory information among a diverse user set, such as physicians, patients, analysts, and technicians. However, due to the rapidly changing pace of laboratory infrastructure and the diversity of laboratory techniques, there exists a need for a highly configurable and adaptable LIMS system to increase the lifespan of laboratory equipment and reduce the occurrence of equipment upgrades. Therefore, a method and system for an automatic diagnostic laboratory for high throughput is provided.

### BRIEF SUMMARY OF THE INVENTION

By utilizing the disclosed invention, the above deficiencies are reduced or eliminated. In some embodiments, a method for managing information in a laboratory is performed. In some embodiment, the method comprises: receiving sample information, the sample information including at least one sample identifier and sample order information; sending movement information to one or more robotics units based on at least the sample identifier; performing, on at least one identified sample, one or more analytical functions to generate results data; and organizing the results data based on the sample order information.

In some embodiments, sample order information includes a disease panel order. In some embodiments, organizing the results data further comprises: rendering a graphical user interface to allow a user to manipulate the results data; and storing updated results data based on one or more user inputs. In some embodiments, the method further comprises receiving a user command including correction information for a sample plate; and storing positional information based on the correction information. In some embodiments, the method further comprises: maintaining one or more variant call format (VCF) files based on the sample information. In some embodiments, sending movement information to one or more robotics units comprises: communicating with at least one vision system in order to identify a physical location of at least one sample. In some embodiments, the method further comprises: communicating with at least one pneumatics system in order to control the movement of at least one sample.

In some embodiments, a system for managing information in a laboratory is utilized. In some embodiments, the system comprises: a display; one or more processors; and a memory storing one or more programs, wherein the one or more programs include instructions configured to be executed by the one or more processors, causing the one or more processors to perform operations comprising: receiving sample information, the sample information including at least one sample identifier and sample order information; sending movement information to one or more robotics units based on at least the sample identifier; performing, on at least one identified sample, one or more analytical functions to generate results data; and organizing the results data based on the sample order information.

In some embodiments, the sample order information includes a disease panel order. In some embodiments, instructions for organizing the results data further comprise instructions for: rendering a graphical user interface to allow a user to manipulate the results data; and storing updated results data based on one or more user inputs. In some embodiments, the one or more programs further comprise instructions for: receiving a user command including correction information for a sample plate; and storing positional information based on the correction information. In some embodiments, the one or more programs further comprise instructions for: maintaining one or more variant call format (VCF) files based on the sample information. In some embodiments, instructions for sending movement information to one or more robotics units further comprise instructions for: communicating with at least one vision system in order to identify a physical location of at least one sample. In some embodiments, instructions for sending movement information to one or more robotics units further comprise instructions for: communicating with at least one pneumatics system in order to control the movement of at least one sample.

In some embodiments, a non-transitory computer readable storage medium is utilized. In some embodiments, the storage medium has instructions stored thereon, the instructions, when executed by one or more processors, cause the processors to perform one or more operations. In some embodiments, the storage medium comprises instructions for: receiving sample information, the sample information including at least one sample identifier and sample order information; sending movement information to one or more robotics units based on at least the sample identifier; performing, on at least one identified sample, one or more analytical functions to generate results data; and organizing the results data based on the sample order information.

In some embodiments, the sample order information includes a disease panel order. In some embodiments, the storage medium comprises instructions for: rendering a graphical user interface to allow a user to manipulate the results data; and storing updated results data based on one or more user inputs. In some embodiments, the storage medium comprises instructions for: receiving a user command including correction information for a sample plate; and storing positional information based on the correction information. In some embodiments, the storage medium comprises instructions for: maintaining one or more variant call format (VCF) files based on the sample information. In some embodiments, instructions for sending movement information to one or more robotics further comprise instructions for: communicating with at least one vision system in order to identify a physical location of at least one sample.

### BRIEF DESCRIPTION OF THE DRAWINGS

FIG. 1 illustrates an overview of an automatic diagnostic laboratory and a laboratory information management system.

FIG. 2 illustrates a detailed view of a laboratory execution system for facilitating laboratory automation.

FIG. 3 illustrates a detailed view of a laboratory information management system for facilitating laboratory automation.

FIG. 4 illustrates a general computing system in which one or more systems may be implemented.

FIG. 5 illustrates an exemplary workflow diagram for sample processing.

FIG. 6 illustrates an exemplary process for controlling a robotics unit to transport one or more sample tubes.

FIG. 7 illustrates a robotic system for managing automatic laboratory processes.

FIG. 8 illustrates an angled view of a robotic arm.

### DETAILED DESCRIPTION OF THE INVENTION

In general, the invention provides for automation and laboratory information management, and may be embodied as a system, method, or computer program product. Furthermore, the present invention may take the form of an entirely software embodiment, entirely hardware embodiment, or a combination of software and hardware embodiments. Even further, the present invention may take the form of a computer program product contained on a computer-readable storage medium, where computer-readable code is embodied on the storage medium. In another embodiment, the present invention may take the form of computer software implemented as a service (SaaS). Any appropriate storage medium may be utilized, such as optical storage, magnetic storage, hard disks, or CD-ROMs.

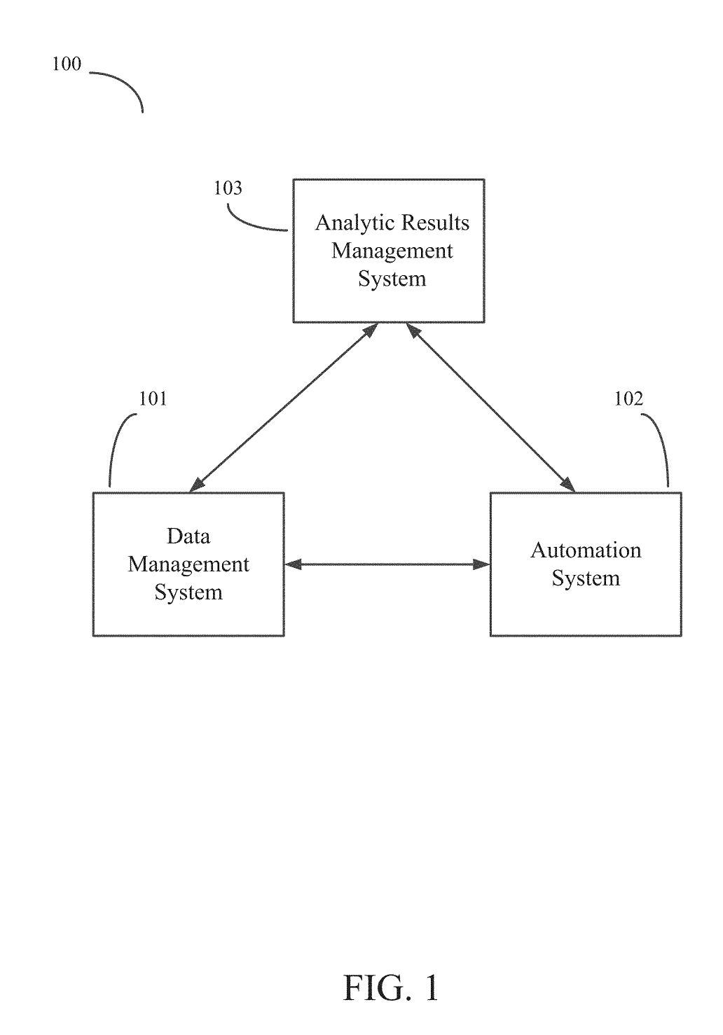

FIG. 1 illustrates an overview of system 100 for an automatic diagnostic laboratory and laboratory information management system (hereinafter “LIMS”). System 100 includes a data management system 101, automation system 102, and analytics results management system (ARMS) 103. In general, data management system 101 is a centralized database tool for maintaining information pertaining to the LIMS system, such as maintaining laboratory tests, diagnostics, equipment, personnel, and the like. In one embodiment, data management system 101 is dynamically updated and facilitates the management of information among other components of the LIMS system, such as automation system 102 and ARMS 103.

Automation system 102 generally provides for the management of laboratory workflow, and may permit one or more users to create and deploy custom laboratory workflow processes. For example, automation system 102 may provide functionality for a user to create a graphical diagram to model different laboratory equipment and diagnostics, and may permit the user to customize the timing, decision-making, and other test variables of laboratory analytics. Automation system 102 may further provide functionality to permit a user to deploy one or more workflow processes based on user-generated diagrams, and such workflow processes may be modified dynamically by the user. Furthermore, automation system 102 may include hardware and software components for interfacing with laboratory equipment, such as robotics units, conveyor systems, sample repositories, climate control systems, (e.g., lighting and temperature), pneumatic systems, audio/video systems, etc.

In one embodiment, automation system 102 may include hardware and/or software for enabling one or more robotics units to perform movements related to testing laboratory samples, such as mixing, shaking, heating, cooling, picking, and/or placing or samples. For example, automation system 102 may generate and send commands to the one or more robotics units to allow the robotics units to move in three-dimensional space. Such commands may also permit the one or more robotics units to interface with a pneumatics system to utilize pressurized air for grasping and releasing one or more samples. In one embodiment, the samples may be contained in a test tube, vial, or similar container. Automation system 102 may further be configured to generate and send commands to the one or more robotics units to allow the robotics units to remove and/or replace a lid on the top of a container. For example, the one or more robotics units may be equipped with machinery capable of sensing a test tube lid, and further capable of removing the test tube lid by one or more robotic motions. Similarly, the one or more robotics units may be equipped with machinery to sense a test tube without a lid, and may perform one or more robotic motions to place and seal the test tube with a lid, for example.

ARMS 103 generally provides a system for dynamically rendering and organizing laboratory information, including but not limited to information such as diagnostic results, quality control metrics, historical test data, sample genotypes, and the like. For example, ARMS 103 may facilitate the generation of interactive data visualizations to permit one or more users to effectively oversee laboratory chemistry, algorithms, and products. ARMS 103 may also permit one or more users to perform complex analytical functions, such as analyze and manipulate quality control constraints, synthesize raw test data, and manually correct test results.

In one embodiment, one or more components of the data management system 101, automation system 102, and/or ARMS 103 may be maintained at a location local to the laboratory and associated equipment (e.g., a server room). In another embodiment, one or more components of the data management system 101, automation system 102, and/or ARMS 103 may be maintained at a location remote from the laboratory and associated equipment (e.g., a “cloud-based” system). In yet another embodiment, one or more components of the data management system 101, automation system 102, and/or ARMS 103 may be maintained in a combination of local and remote locations.

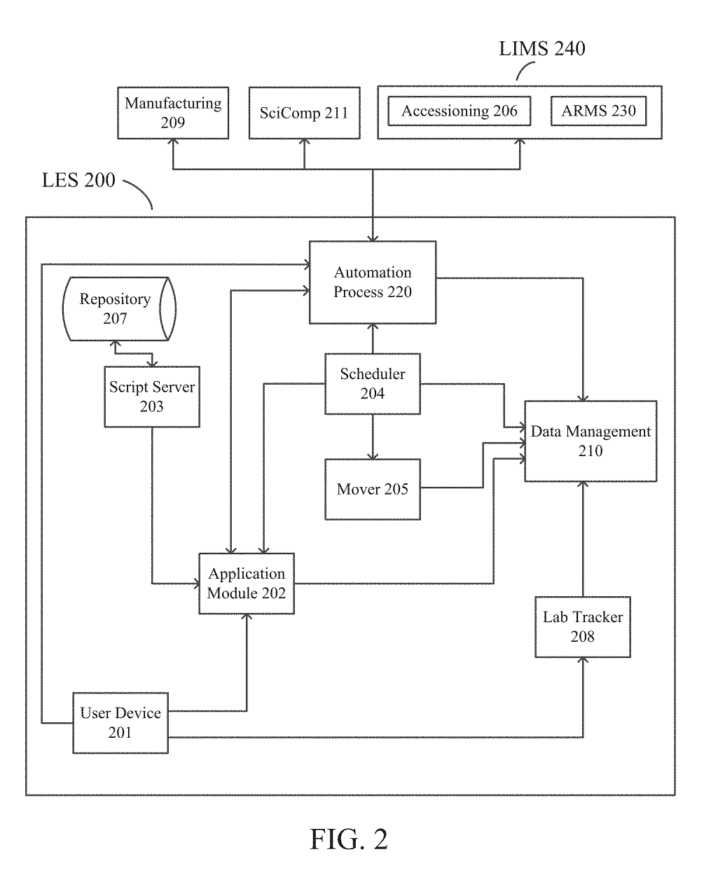

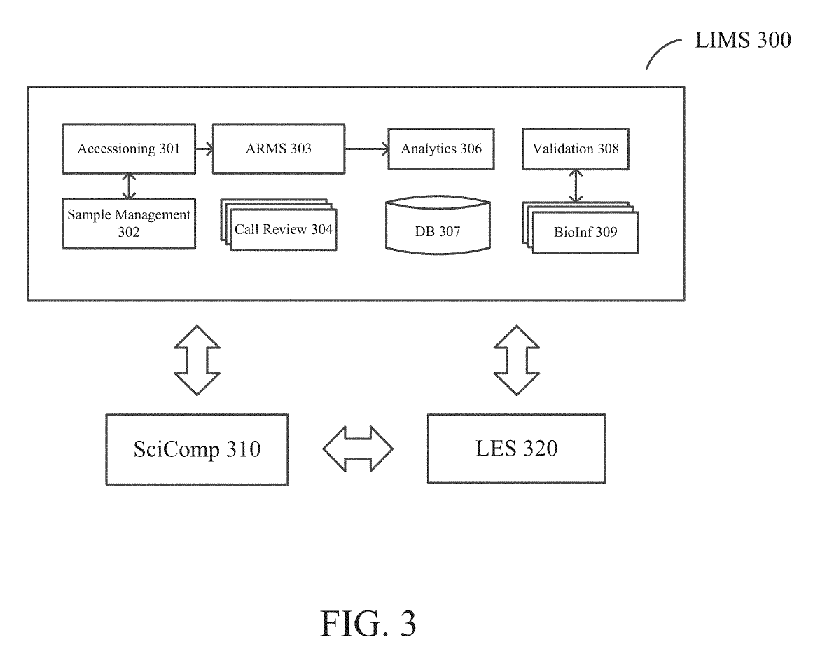

FIG. 2 illustrates a detailed view of a laboratory execution system (LES) 200. LES 200 may include a data management tool 210 and an automation process 220. Furthermore, LES 200 may communicate with a LIMS module 240. In one embodiment, LIMS module 240 may include at least an accessioning module 206 and an analytic results management system (ARMS) 230, which are discussed in more detail with respect to FIG. 3. FIG. 2 further depicts user device 201 and application module 202, which will now be described. User device 201 may permit a user to interact with LES 200 and thus facilitate user interaction with each of the data management tool 210, automation process 220, and ARMS 230, and/or other associated systems. User device 201 may communicate with application module 202 in order to perform one or more functions as described herein.

In one embodiment, application module 202 may be an application programming interface (API) for performing one or more automated functions. In another embodiment, application module 202 may be a graphical user interface (GUI), whereby a user may instruct LES 200 to perform one or functions such as loading a script, running a diagnostic method, executing a laboratory instrument action, or the like. User device 201 may also interface with LES 200 by direct interaction with other components of the system. For example, user 201 may provide a command directly to scheduler 204 for fixing execution time errors.

In another embodiment, lab tracker 208 facilitates physical location management of one or more robotics units. For example, lab tracker 208 may be configured as a database which stores positional information of all physical objects for a given point in time. Lab tracker 208 may also receive information from other components in LES 200. For example, user 201 may provide a command to lab tracker module 208 for fixing a plate tracking error.

FIG. 2 further depicts automation process 220, which may provide workflow management of sample plates, samples, and associated data. For example, automation process 220 may provide information regarding available plates to application module 202, or may otherwise indicate the availability of system resources to application module 202. As another example, automation process 220 may receive reporting information, such as a job completion report, from application module 202. Automation process 220 may also receive seed pipeline information, which may be manually entered by a user and provided directly to the automation process 220 from user device 201. Seed pipeline information may include, for example, information to instantiate new objects for management into the LIMS system. For example, a user may utilize a GUI in order to create research samples, where the research samples are introduced as seed pipeline information into automation process 220.

In another embodiment, automation process 220 may receive seed pipeline information from an accessioning module 206. In yet another embodiment, automation process 220 may receive query information from ARMS 230, for example, a query regarding results to be displayed. Automation process 220 may further receive query information from scheduler 204, for example, a query regarding a pending job. Furthermore, automation process 220 may provide data management tool 210 with data validation information and information regarding data queries.

Furthermore, FIG. 2 shows data management tool 210, which will now be described. Data management tool 210 may be configured to integrate quantitative data, track sample barcodes, and manage overall workflow of LES 200. In one embodiment, data management tool 210 may receive information regarding a report operation from application module 202. In another embodiment, data management tool 210 may receive a report operation from mover module 205. Furthermore, data management tool 210 may receive a command to fix plate tracking errors from a user via lab tracker module 207. In yet another embodiment, data management tool 210 may receive, from scheduler 204, a query regarding stateful data. In one example, such a query pertains to seal, spin, or location information.

FIG. 2 further depicts script server 203 and repository 207, which will now be described. In one embodiment, script server 203 may communicate with a version control system (VCS) repository 207 in order to obtain one or more software scripts for use in operating LES 200. VCS repository 207 may be maintained by known repositories such as “Github,” or any other appropriate VCS repository service, as will be appreciated by one of ordinary skill in the art. In one embodiment, script server 203 may obtain software scripts from VCS repository 207, and may further push one or more software scripts to application module 202. Script server 203 may be further configured to deploy scripts and manage script metadata.

Scheduler 204 may be configured to automate scheduling and execute applications. For example, scheduler 204 may include at least one software module such as script compiler, scheduler, and/or executor. In one embodiment, scheduler 204 may provide application module 202 with one or more commands for performing an action, or may further provide application module 202 with a query for an API function. In another embodiment, scheduler 204 may be configured to initiate and/or deliver one or more queries for an API function, and may be further configured to initiate and/or deliver one or more queries regarding stateful data. In another embodiment, scheduler 204 may be configured to initiate and/or deliver one or more queries regarding a pending job. In yet another embodiment, scheduler 204 may be configured to receive a command to fix execution time errors.

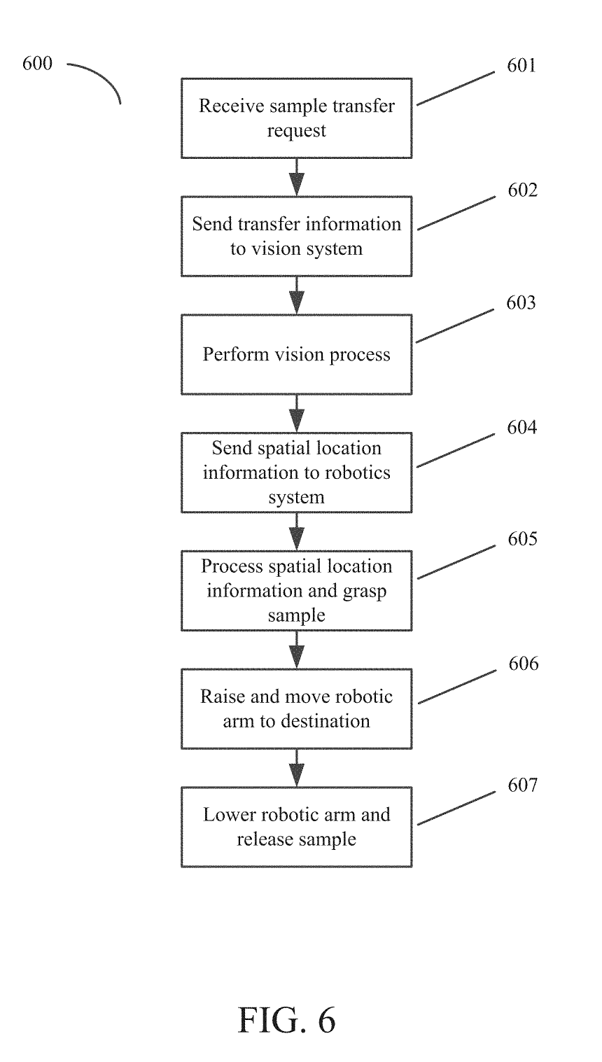

Mover application 205 may be configured to communicate with one or more robotics units within a laboratory environment. For example, mover application 205 may facilitate the directing of the one or more robotics units to perform one or more movements in three-dimensional space. Mover application 205 may send instructions to the one or more robotics units regarding a movement, path, direction, or other information relating to three-dimensional space in which the one or more robotics units may perform any number of movements. In another embodiment, scheduler 204 may provide mover module 205 with one or more commands for performing a move, such as, for example, robotic movements described in detail with respect to FIG. 6.

Additionally, LES 200 may be configured to communicate with manufacturing module 209. In one embodiment, manufacturing module 209 is configured to provide LES 200 with information related to sample components, such as plastic, reagents, and the like. For example, manufacturing module 209 may assist in identifying sample components which are introduced into LES 200. In another embodiment, manufacturing module 209 may be configured to declare and generate barcode labels for one or more sample plates and sample tubes.

LES 200 may further communicate with SciComp module 211. In one embodiment, SciComp module 211 may facilitate overall automation within the LIMS system by managing the processing of all main stages, including but not limited to (i) physical sample acquisition, (ii) sequencing, (iii) raw data generation, (iv) data analysis, and (v) transfer of analyzed data to ARMS. In one example, SciComp module 211 may assist automation process 220 by querying automation process 200 for information pertaining to a next job to process. SciComp module 211 may further include components such a script server and/or scheduler for maintaining efficient job workflow. In one embodiment, SciComp module 211 may perform the necessary data analytics tasks of the LIMS system, and may run the necessary algorithms to automatically produce patient variant calls from raw data to analyzed data. In some embodiments, SciComp module 211 and/or automation process 220 can operate independently or together to concurrently perform workflows for different kinds of samples (e.g., concurrently perform workflows for research samples and production samples, where production samples have different testing processes applied to them than research samples, and where such processes are being performed on the samples concurrently). Thus, SciComp module 211 and/or automation process 220 can differentiate between production samples (e.g., tagged with a production identifier) and research samples (e.g., tagged with a research identifier) and process the samples accordingly. Existing robotics systems are not able to process research and production samples concurrently; thus, the system of the disclosure is an improvement on existing robotics systems.

In some embodiments, SciComp module 211 and/or automation process 220 can operate independently or together to automatically determine which subset of tests to perform on one or more samples based on a disease panel order for the one or more samples and/or the test(s) (e.g., assays or test protocols) that system 100 is currently configured to perform. For example, application module 202 can be configured to receive an input (e.g., via user device 201) to implement one or more specified disease panels for one or more specified samples. Automation process 220 and/or SciComp module 211 (in some embodiments, in conjunction with application module 202, script server 203 and/or repository 207) can, based on the specified disease panels, determine which test(s) must be performed on the specified samples in order to satisfy the specified disease panels (e.g., in order to determine information about the samples required for the disease panels). In some embodiments, the specified disease panels can be satisfied with alternative tests; as such, in some embodiments, automation process 220 and/or SciComp module 211 can determine which of those alternative tests (e.g., a subset of those alternative tests) to perform on the specified samples in order to satisfy the specified disease panels. Further, in some embodiments, system 100 may only be configured to perform a subset of those alternative tests at the current time (e.g., alternative tests A, B and C may satisfy the specified disease panel, but currently, system 100 may only be configured to perform tests A and C). In such embodiments, automation process 220 and/or SciComp module 211 can, from the requested disease panels, determine which alternative tests to perform on the specific samples to select tests that system 100 is currently configured to perform that will also satisfy the requested disease panels.

Although only one instance of each module is listed on FIG. 2 (e.g. one scheduler 204 and one mover 205), LES 200 may include one or more instances of any such module. For example, there may be two or more instances of scheduler 204, which are each associated with a specific process or device within the laboratory environment.

FIG. 3 illustrates a detailed depiction of laboratory information management system (LIMS) 300. In one embodiment, LIMS 300 includes an accessioning module 301 and sample management module 302. Accessioning module 301 may be configured to record the arrival of a sample and instantiate the arrival of the sample within one or more databases. For example, accessioning module 301 may be configured to send a first set of information to ARMS 303. The first set of information may include, for example, information pertaining to a disease panel order and/or information indicating one or more required assays/test protocols to be performed on the sample (e.g., an assay order). Sample management module 302 may be configured to communicate with accessioning module 301 in the organization of one or more samples to be seeded to ARMS 303. Analytics module 306 may receive one or more outputs from ARMS 303, such as results pertaining to a disease panel order. LIMS 300 may further include validation module 308 and bioinformation module 309. Validation module 308 and bioinformation module 309 may each be configured to assist in the development of sample assays for testing.

As depicted in FIG. 3, LIMS 300 may further communicate with LES 310 and SciComp 320, as discussed with respect to FIG. 2. LIMS 300 may further include a call review module 304, which may be configured to provide processing techniques to review and modify variant call processing data. LIMS 300 may further include a database module 307 to store information relating to samples and associated test data, as used within LIMS 300.

ARMS 303 may be further configured as a database containing genotypes for samples. For example, ARMS 303 may be configured to process, maintain, and deliver information regarding genotyping data based on one or more Variant Call Format (VCF) files. As will be appreciated by one of ordinary skill in the art, a VCF file is a standardized text file format for representing and storing gene sequence variations. In one embodiment, ARMS 303 may provide a results query to an automation process on LES 320. For example, a results query may be utilized to determine which results are capable of being displayed.

In another embodiment, ARMS 303 includes functionality for generating a GUI, where the GUI provides a user with real-time data corresponding to laboratory diagnostics and analysis for one or more samples. The GUI may permit the user to perform a plurality of functions, including but not limited to quality control (QC) monitoring and adjustment, sample history generation, manual tagging of samples, and the ability to manually pass or fail a given sample. ARMS 303 may include functionality for generating custom diagnostics reports, including the generation of graphs, tables, spreadsheets, plots, diagrams, and/or other visualization to enable efficient data interpretation.

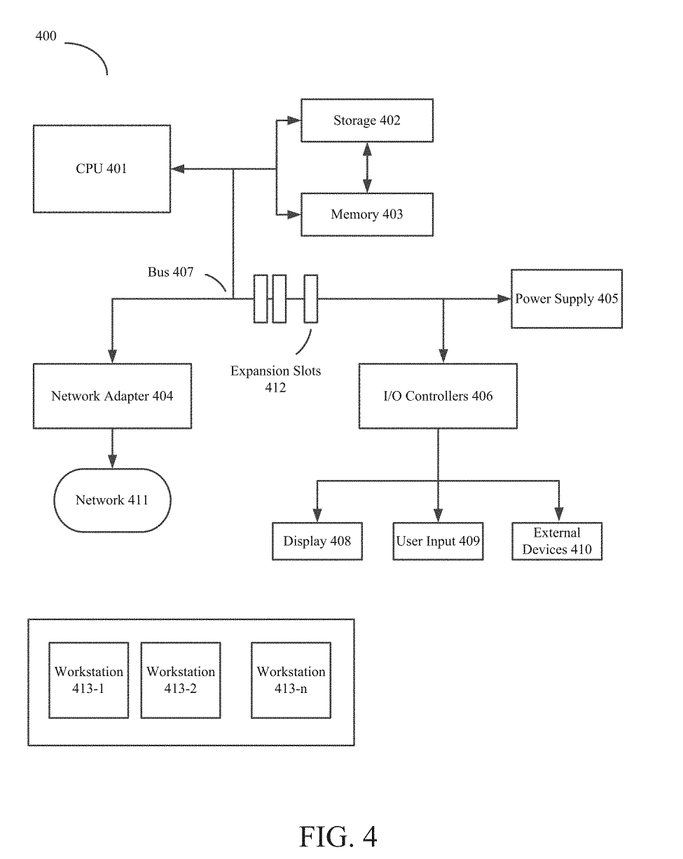

FIG. 4 illustrates a general purpose computing system 400 in which one or more systems, as described herein, may be implemented. System 400 may include, but is not limited to known components such as central processing unit (CPU) 401, storage 402, memory 403, network adapter 404, power supply 405, input/output (I/O) controllers 406, electrical bus 407, one or more displays 408, one or more user input devices 409, and other external devices 410. It will be understood by those skilled in the art that system 400 may contain other well-known components which may be added, for example, via expansion slots 412, or by any other method known to those skilled in the art. Such components may include, but are not limited, to hardware redundancy components (e.g., dual power supplies or data backup units), cooling components (e.g., fans or water-based cooling systems), additional memory and processing hardware, and the like.

System 400 may be, for example, in the form of a client-server computer capable of connecting to and/or facilitating the operation of a plurality of workstations or similar computer systems over a network. In another embodiment, system 400 may connect to one or more workstations over a intranet or internet network, and thus facilitate communication with a larger number of workstations or similar computer systems. Even further, system 400 may include, for example, a main workstation or main general purpose computer to permit a user to interact directly with a central server. Alternatively, the user may interact with system 400 via one or more remote or local workstations 413. As will be appreciated by one of ordinary skill in the art, there may be any practical number of remote workstations for communicating with system 400.

CPU 401 may include one or more processors, for example Intel® Core™ i7 processors, AMD FX™ Series processors, or other processors as will be understood by those skilled in the art. CPU 401 may further communicate with an operating system, such as Windows NT® operating system by Microsoft Corporation, Linux operating system, or a Unix-like operating system. However, one of ordinary skill in the art will appreciate that similar operating systems may also be utilized. Storage 402 may include one or more types of storage, as is known to one of ordinary skill in the art, such as a hard disk drive (HDD), solid state drive (SSD), hybrid drives, and the like. In one example, storage 402 is utilized to persistently retain data for long-term storage. Memory 403 may include one or more types memory as is known to one of ordinary skill in the art, such as random access memory (RAM), read-only memory (ROM), hard disk or tape, optical memory, or removable hard disk drive. Memory 403 may be utilized for short-term memory access, such as, for example, loading software applications or handling temporary system processes.

As will be appreciated by one of ordinary skill in the art, storage 402 and/or memory 403 may store one or more computer software programs. Such computer software programs may include logic, code, and/or other instructions to enable processor 401 to perform the tasks, operations, and other functions as described herein, and additional tasks and functions as would be appreciated by one of ordinary skill in the art. Operating system 402 may further function in cooperation with firmware, as is well known in the art, to enable processor 401 to coordinate and execute various functions and computer software programs as described herein. Such firmware may reside within storage 402 and/or memory 403.

Moreover, I/O controllers 406 may include one or more devices for receiving, transmitting, processing, and/or interpreting information from an external source, as is known by one of ordinary skill in the art. In one embodiment, I/O controllers 406 may include functionality to facilitate connection to one or more user devices 409, such as one or more keyboards, mice, microphones, trackpads, touchpads, or the like. For example, I/O controllers 406 may include a serial bus controller, universal serial bus (USB) controller, FireWire controller, and the like, for connection to any appropriate user device. I/O controllers 406 may also permit communication with one or more wireless devices via technology such as, for example, near-field communication (NFC) or Bluetooth™. In one embodiment, I/O controllers 406 may include circuitry or other functionality for connection to other external devices 410 such as modem cards, network interface cards, sound cards, printing devices, external display devices, or the like. Furthermore, I/O controllers 406 may include controllers for a variety of display devices 408 known to those of ordinary skill in the art. Such display devices may convey information visually to a user or users in the form of pixels, and such pixels may be logically arranged on a display device in order to permit a user to perceive information rendered on the display device. Such display devices may be in the form of a touch-screen device, traditional non-touch screen display device, or any other form of display device as will be appreciated be one of ordinary skill in the art.

Furthermore, CPU 401 may further communicate with I/O controllers 406 for rendering a graphical user interface (GUI) on, for example, one or more display devices 408. In one example, CPU 401 may access storage 402 and/or memory 403 to execute one or more software programs and/or components to allow a user to interact with the system as described herein. In one embodiment, a GUI as described herein includes one or more icons or other graphical elements with which a user may interact and perform various functions. For example, GUI 407 may be displayed on a touch screen display device 408, whereby the user interacts with the GUI via the touch screen by physically contacting the screen with, for example, the user's fingers. As another example, GUI may be displayed on a traditional non-touch display, whereby the user interacts with the GUI via keyboard, mouse, and other conventional I/O components 409. GUI may reside in storage 402 and/or memory 403, at least in part as a set of software instructions, as will be appreciated by one of ordinary skill in the art. Moreover, the GUI is not limited to the methods of interaction as described above, as one of ordinary skill in the art may appreciate any variety of means for interacting with a GUI, such as voice-based or other disability-based methods of interaction with a computing system. One or more of the above-described modules (e.g., LIMS module, accessioning module, application module, lab tracker module, mover module and/or etc.) can be implemented by one or more components of system 400 (e.g., CPU 401 executing instructions stored in memory 403).

Moreover, network adapter 404 may permit device 400 to communicate with network 411. Network adapter 404 may be a network interface controller, such as a network adapter, network interface card, LAN adapter, or the like. As will be appreciated by one of ordinary skill in the art, network adapter 404 may permit communication with one or more networks 411, such as, for example, a local area network (LAN), metropolitan area network (MAN), wide area network (WAN), cloud network (IAN), or the Internet.

One or more workstations 413 may include, for example, known components such as a CPU, storage, memory, network adapter, power supply, I/O controllers, electrical bus, one or more displays, one or more user input devices, and other external devices. Such components may be the same, similar, or comparable to those described with respect to system 400 above. It will be understood by those skilled in the art that one or more workstations 413 may contain other well-known components, including but not limited to hardware redundancy components, cooling components, additional memory/processing hardware, and the like.

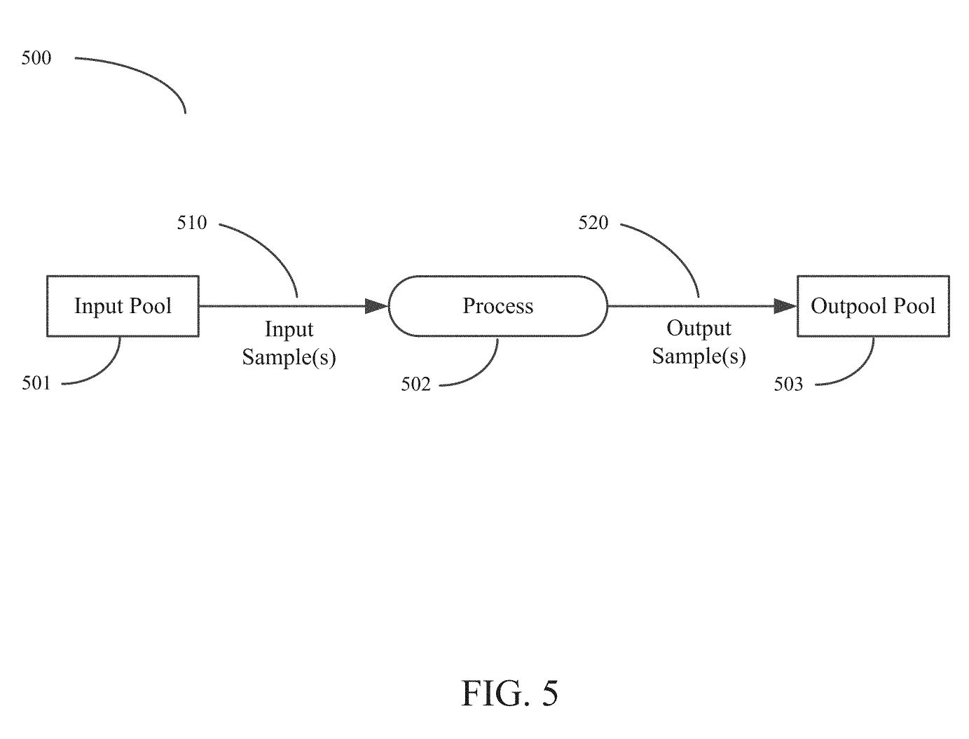

FIG. 5 illustrates an exemplary laboratory process 500 facilitated by, for example, automation process 220 in FIG. 2. In one embodiment, automation process 220 provides a user with the ability to create lab workflow processes in order to maintain sample queues for diagnostics and analysis. For example, a user may create one or more graphical objects on a GUI display, where the objects may represent one or more laboratory states, decisions, inputs, outputs, or other conditions to model a laboratory process. A resulting laboratory process may be created based on the one or more graphical objects created by the user, such as, for example, a process as depicted in FIG. 5.

In one embodiment, process 500 includes input pool object 501, which may represent, for example, one or more polymerase chain reaction (PCR) plates. Samples from the input pool may be scheduled to undergo one or more tests, diagnostics, or other laboratory processes 502. For example, samples within the one or more PCR plates may undergo a process for DNA amplification. Arrow 510 may represent the transfer of one PCR plate 501 to amplification process 502, for example. Arrow 520 may represent a successful output of amplification process 502, such as, for example, one amplified PCR plate. Output pool 503 may represent, for example, one or more amplified PCR plates. Arrow 520 may therefore represent the transfer of one amplified PCR plate to output pool object 503. Although only one input, one process, and one output are depicted in process 500, it will be appreciated that any number of inputs, outputs, processes, transfers, or other laboratory functions may be represented by such a graphical diagram, and that the invention is not limited to the exemplary process depicted in FIG. 5.

FIG. 6 illustrates an exemplary transfer process 600 for controlling a robotics unit to transport one or more sample tubes. Transfer process 600 may be facilitated at least in part by automation process 220 as described in FIGS. 1 and 2. For example, automation process 220 may communicate with hardware and software processes associated with one or more robotics, vision, and/or pneumatic systems in order to perform transfer process 600. In one embodiment, transfer process 600 is utilized to transfer at least one sample from an origin location to a destination location by using at least one robotics unit coupled with vision and pneumatics systems.

Transfer process 600 may begin at step 601, where automation process may receive a sample transfer request. Such request may be, for example, a manual request entered by a user, or may be an automated request initiated by a pre-scheduled workflow process. In one embodiment, the request includes information identifying at least one sample barcode corresponding to a current sample, and may further include information identifying a destination location for transferring the sample associated with the sample barcode from an origin location to the destination location.

At step 602, automation process may send transfer information to a vision system in order to identify the spatial location of the identified sample. In one embodiment, the vision system performs a vision matching process at step 603 to identify if a matching barcode exists within the vision system's viewing area. If a matching barcode is found, the vision system may send corresponding spatial location information to robotics system at step 604. Such spatial location information may correspond to sample location information discovered by the vision system when identifying matching barcode in step 603. The spatial location information may be in a form readable by robotics unit in order to permit the robotics unit to identify a three dimensional location in space corresponding to the physical sample identified.

At step 605, the robotics unit may receive and process the spatial location information, and may further grasp the identified sample. For example, the robotics unit may utilize the spatial location information to move a robotic arm to a location corresponding to a position directly above the identified sample. The robotic arm may then be lowered to a location near the sample, and the arm may grasp the sample by utilizing, for example, a pneumatic system. In one example, the sample is contained in a test tube which is grasped by a robotic arm, where a pneumatic system generates a vacuum in order to grip the test tube.

At step 606, the robotic arm may be raised while grasping the sample, and the robotic arm may be moved to a location corresponding to a destination location as received in the sample transfer request. At step 607, the robotic arm may lower the sample onto a location corresponding to the desired location, and may release the sample from the robotic grip by performing one or more pneumatic processes via the pneumatic system. For example, the pneumatic system may release the grip on the sample by discharging the vacuum and briefly expelling air near the sample.

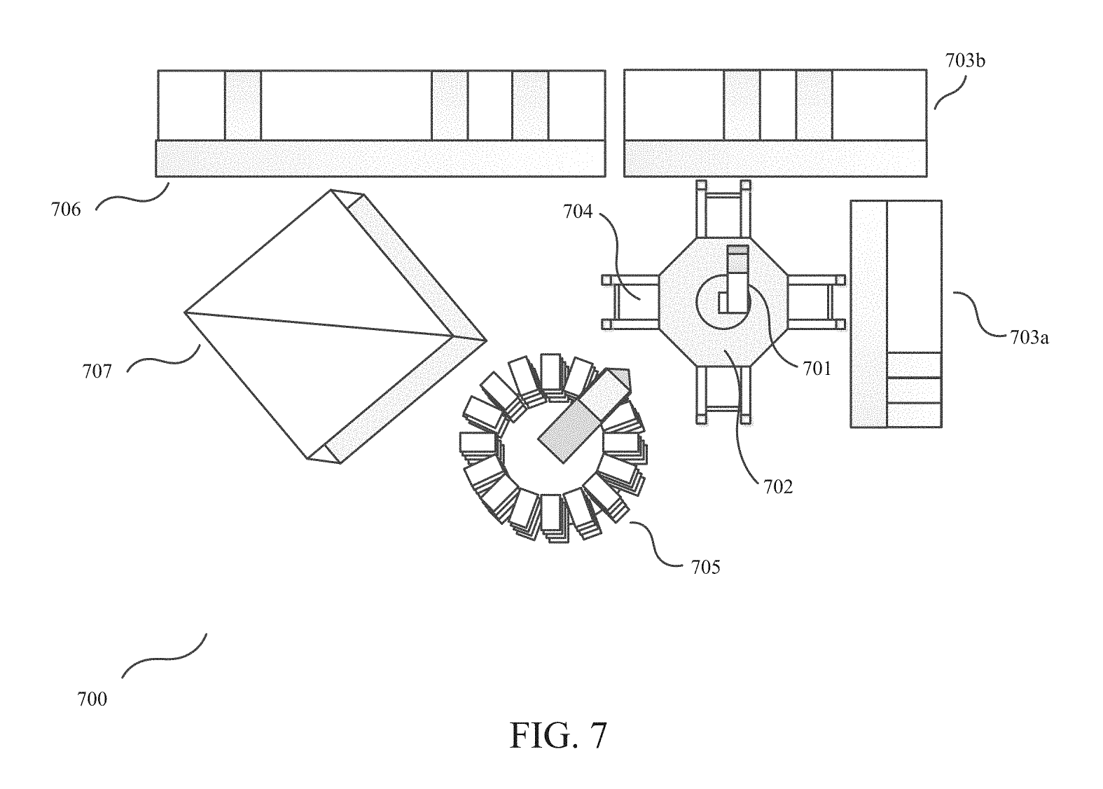

FIG. 7 illustrates a robotic system 700 for managing automatic laboratory processes. In one embodiment, robotic system 700 includes a robotic arm 701 for facilitating the movement of one or more samples. For example, robotic arm 701 may be configured to grasp a test tube containing a sample, and transport the test tube from a first location to a second location. In another example, robotic arm 701 may be configured to grasp a sample rack, and transport the sample rack from a first location to a second location. A sample rack may contain one or more samples, and may be stored, for example, in a sample rack repository 705. In one embodiment, sample rack repository 705 may contain one or more sample racks and may facilitate efficient storage and retrieval of one or more sample racks.

In one embodiment, robotic arm 701 may further be affixed to a robotic arm base 702, and may be configured to rotate in a 360 degree motion about the laboratory environment. For example, robotic arm 701 may extend from a first position, such as the position depicted in FIG. 7, to a second position, such as a position extending into a first liquid handling apparatus 703a. Furthermore, robotic arm 701 may, for example, retract from the extended position in first liquid handling apparatus 703a and return to the position as depicted in FIG. 7. Furthermore, robotic arm 701 may retract from the position in first liquid handling apparatus 703a, and then extend to a position within a second liquid handling apparatus 703b. In one embodiment, the robotic arm may perform various movements within liquid handling apparatus 703a and liquid handling apparatus 703b in order to facilitate various sample test procedures.

In another embodiment, robotic arm 701 may be configured to transport one or more samples and/or sample racks from sample rack repository 705 to liquid handling apparatus 703a or liquid handling apparatus 703b. Robotic arm 701 may further be configured to return one or more samples and/or sample racks from liquid handling apparatus 703a or liquid handling apparatus 703b to sample rack repository 705, for example. Furthermore, although only two liquid handling apparatus 703a and 703b are depicted in FIG. 7, one will appreciate that additional liquid handling apparatus may be deployed within the laboratory environment, and that robotic arm 701 may extend into other such areas within the reach of robotic arm 701.

In yet another embodiment, robotic arm 701 may be surrounded by one or more sensors 704. Sensors 704 may, for example, detect specific motions within an area surrounding robotic arm 701, such as a predefined motion detection area. In one embodiment, the motion detection area may be defined by a spherical or semi-spherical region centered at or near a coupling point of robotic arm 701 to robotic arm base 702. In another embodiment, the motion detection area may be defined by a spherical or semi-spherical region centered at or near a specific point in space defined by a user. For example, the motion detection area may be dynamically configured and updated by a user, and may define custom three-dimensional areas in space surrounding robotic arm 701.

Sensors 704 may, for example, provide signals to one or more software systems within the laboratory environment in order to prevent robotic arm 701 from moving into specific areas within the laboratory environment. In one example, sensors 704 may be configured to detect movements associated with a user or other object within a specified motion detection area near robotic arm 701. If sensors 704 detect such motions, sensors 704 may send one or more alarm signals to software systems associated with robotic arm 701 in order to cease all movements of robotic arm 701. Sensors 704 may be configured to, for example, send signals to software systems associated with robotic arm 701 in order to resume movements of robotic 701 upon the sensors 704 detecting that any such user, object, or other event causing the alarm signals is no longer within the motion detection area. In another embodiment, sensors 704 and robotic arm 701 may remain disabled after the alarm signal until a predefined user restart process is initiated and completed. Upon completion of such user restart process, the robotic arm 701 and sensors 704 may, for example, resume normal operations.

In another embodiment, robotic system 700 includes an additional liquid handling apparatus 706 having a robotics unit configured for automated DNA extraction. Liquid handling apparatus 706 may be configured to handle multiple tube sizes and/or multiple sample types. For example, liquid handling apparatus 706 may be configured to handle either a 4 mm tube size or a 6 mm tube size. In another example, liquid handling apparatus 706 may be configured to handle either a blood sample or a saliva sample. In another embodiment, robotic system 700 includes a robotic refrigerator 707, which may be configured to store and retrieve sample plates of one or more different sizes. Robotic refrigerator 707 may be further configured, for example, to allow for human override to permit manual access to the contents within robotic refrigerator 707.

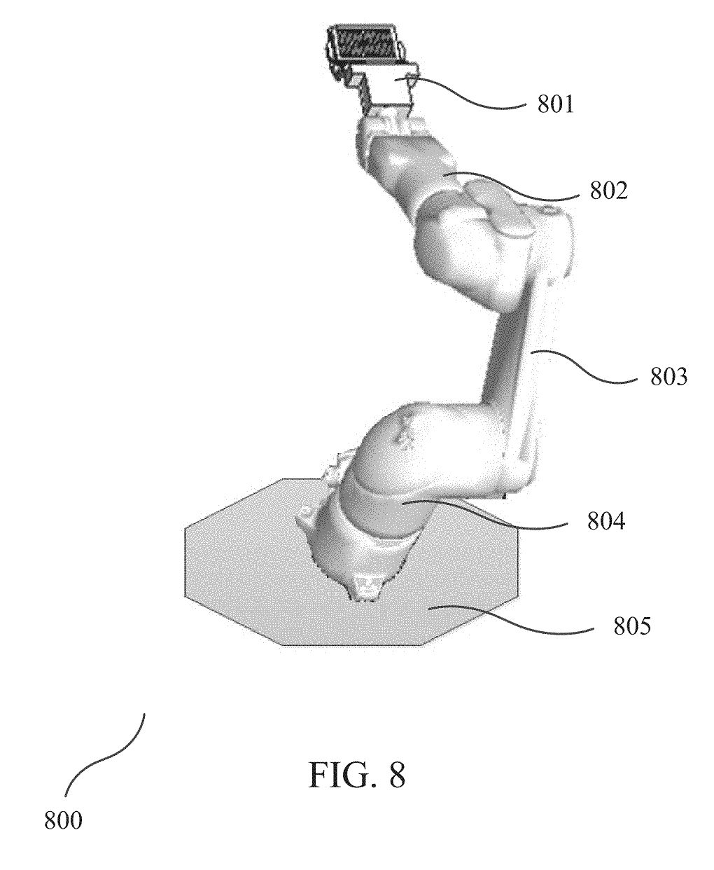

FIG. 8 illustrates an angled view of a robotic arm 800, for example, a robotic arm such as robotic arm 701 in FIG. 7. As depicted in FIG. 8, robotic arm 800 includes a sample handling portion 801, a first solid section 802, a second solid section 803, a third solid section 804, and a robotic arm base 805. In one embodiment, sample handling portion 801 is connected to the first solid section 802. In another embodiment, first solid section 802 is connected at one end to sample handling portion 801, and is connected at another end to second solid section 803. In yet another embodiment, second solid section 803 is connected at one end to first solid section 802, and is connected at another end to third solid section 804. In yet another embodiment, third solid section 804 is connected at one end to second solid section 803, and is connected at another end to robotic arm base 805.

### EXAMPLES

Therefore, examples of the disclosure include:

Example 1. A method for managing information in a laboratory, the method comprising: receiving sample information, the sample information including at least one sample identifier and sample order information; sending movement information to one or more robotics units based on at least the sample identifier; performing, on at least one identified sample, one or more analytical functions to generate results data; and organizing the results data based on the sample order information.

Example 2. The method of Example 1, wherein the sample order information includes a disease panel order.

Example 3. The method of any one of Examples 1-2, wherein the sample order information includes an assay order.

Example 4. The method of any one of Examples 1-3, wherein the sample order information includes an indication that the at least one identified sample is for research or for production.

Example 5. The method of any one of Examples 1-4, wherein organizing the results data further comprises: rendering a graphical user interface to allow a user to manipulate the results data; and storing updated results data based on one or more user inputs.

Example 6. The method of any one of Examples 1-5, further comprising: receiving a user command including correction information for a sample plate; and storing positional information based on the correction information.

Example 7. The method of any one of Examples 1-6, further comprising: maintaining one or more variant call format (VCF) files based on the sample information.

Example 8. The method of any one of Examples 1-7, wherein sending movement information to one or more robotics units comprises: communicating with at least one vision system in order to identify a physical location of at least one sample.

Example 9. The method of any one of Examples 1-8, further comprising: communicating with at least one pneumatics system in order to control movement of at least one sample.

Example 10. The method of any one of Examples 1-9, further comprising: dynamically routing samples from a sample acquisition stage to a sequencing stage, wherein an automation process facilitates the dynamic routing based on stateful data and defined workflow processes.

Example 11. The method of any one of Examples 1-10, further comprising: retrieving stateful data and defined workflow processes; generating one or more robotic move commands based on at least the stateful data and defined workflow processes; communicating the one or more robotic move commands to the one or more robotics units in order to achieve high throughput sequencing.

Example 12. A system for managing information in a laboratory, the system comprising: a display; one or more processors; and a memory storing one or more programs, wherein the one or more programs include instructions configured to be executed by the one or more processors, causing the one or more processors to perform operations comprising: receiving sample information, the sample information including at least one sample identifier and sample order information; sending movement information to one or more robotics units based on at least the sample identifier; performing, on at least one identified sample, one or more analytical functions to generate results data; and organizing the results data based on the sample order information.

Example 13. The system of Example 12, wherein the sample order information includes a disease panel order.

Example 14. The system of any one of Examples 12-13, wherein instructions for organizing the results data further comprise instructions for: rendering a graphical user interface to allow a user to manipulate the results data; and storing updated results data based on one or more user inputs.

Example 15. The system of any one of Examples 12-14, the one or more programs further comprising instructions for: receiving a user command including correction information for a sample plate; and storing positional information based on the correction information.

Example 16. The system of any one of Examples 12-15, the one or more programs further comprising instructions for: maintaining one or more variant call format (VCF) files based on the sample information.

Example 17. The system of any one of Examples 12-16, wherein instructions for sending movement information to one or more robotics units further comprise instructions for: communicating with at least one vision system in order to identify a physical location of at least one sample.

Example 18. The system of any one of Examples 12-17, wherein instructions for sending movement information to one or more robotics units further comprise instructions for: communicating with at least one pneumatics system in order to control the movement of at least one sample.

Example 19. The system of any one of Examples 12-18, the one or more programs further comprising instructions for: dynamically routing samples from a sample acquisition stage to a sequencing stage, wherein an automation process facilitates the dynamic routing based on stateful data and defined workflow processes.

Example 20. The system of any one of Examples 12-19, the one or more programs further comprising instructions for: retrieving stateful data and defined workflow processes; generating one or more robotic move commands based on at least the stateful data and defined workflow processes; communicating the one or more robotic move commands to the one or more robotics units in order to achieve high throughput sequencing.

Example 21. The system of any one of Examples 12-20, wherein the sample order information includes an assay order.

Example 22. The system of any one of Examples 12-21, wherein the sample order information includes an indication that the at least one identified sample is for research or for production.

Example 23. A non-transitory computer readable storage medium having instructions stored thereon, the instructions, when executed by one or more processors, cause the processors to perform operations comprising: receiving sample information, the sample information including at least one sample identifier and sample order information; sending movement information to one or more robotics units based on at least the sample identifier; performing, on at least one identified sample, one or more analytical functions to generate results data; and organizing the results data based on the sample order information.

Example 24. The storage medium of Example 23, wherein the sample order information includes a disease panel order.

Example 25. The storage medium of any one of Examples 23-24, further comprising instructions for: rendering a graphical user interface to allow a user to manipulate the results data; and storing updated results data based on one or more user inputs.

Example 26. The storage medium of any one of Examples 23-25, further comprising instructions for: receiving a user command including correction information for a sample plate; and storing positional information based on the correction information.

Example 27. The storage medium of any one of Examples 23-26, further comprising instructions for: maintaining one or more variant call format (VCF) files based on the sample information.

Example 28. The storage medium of any one of Examples 23-27, wherein instructions for sending movement information to one or more robotics further comprise instructions for: communicating with at least one vision system in order to identify a physical location of at least one sample.

Example 29. The storage medium of any one of Examples 23-28, further comprising instructions for: communicating with at least one pneumatics system in order to control the movement of at least one sample.

Example 30. The storage medium of any one of Examples 23-29, further comprising instructions for: dynamically routing samples from a sample acquisition stage to a sequencing stage, wherein an automation process facilitates the dynamic routing based on stateful data and defined workflow processes.

Example 31. The storage medium of any one of Examples 23-30, further comprising instructions for: retrieving stateful data and defined workflow processes; generating one or more robotic move commands based on at least the stateful data and defined workflow processes; communicating the one or more robotic move commands to the one or more robotics units in order to achieve high throughput sequencing.

Example 32. The storage medium of any one of Examples 23-31, wherein the sample order information includes an assay order.

Example 33. The storage medium of any one of Examples 23-32, wherein the sample order information includes an indication that the at least one identified sample is for research or for production.

As used herein, the terminology as used throughout the description of the invention is for the purpose of describing particular embodiments only. Such terminology does not limit the scope of the invention in any way. For example, singular forms of “a,” “an” and “the” are intended to include plural forms unless indicated otherwise. Furthermore, terms such as “comprises” or “comprising” specify the presence of indicated features, components, steps, etc., but do not preclude the presence or addition of one or more other features, components, steps, etc. The description may also include the term “in,” which may include “in” and “on” unless clearly indicated otherwise. Furthermore, usage of the term “or” includes both conjunctive and disjunctive meanings, unless clearly indicated otherwise. That is, unless expressly stated otherwise, the term “or” may include “and/or.”

It will be further understood that various modifications to the invention may be made by one skilled in the art without departing from the spirit and scope of the invention as defined in the claims. For example, numerous changes, substitutions, and variations with respect to the systems and methods as described may occur. One of ordinary skill in the art will understand that various alternative embodiments may be employed to practice the invention, and that any feature may be combined with any other feature, whether such features are preferred or not.

1. A method for managing information in a laboratory, the method comprising:
receiving sample information, the sample information including at least one sample identifier and sample order information;
sending movement information to one or more robotics units based on at least the sample identifier;
performing, on at least one identified sample, one or more analytical functions to generate results data; and
organizing the results data based on the sample order information.

2. The method of claim 1, wherein the sample order information includes a disease panel order.

3. The method of claim 1, wherein the sample order information includes an assay order.

4. The method of claim 1, wherein the sample order information includes an indication that the at least one identified sample is for research or for production.

5. The method of claim 1, wherein organizing the results data further comprises:
rendering a graphical user interface to allow a user to manipulate the results data; and
storing updated results data based on one or more user inputs.

6. The method of claim 1, further comprising:
receiving a user command including correction information for a sample plate; and
storing positional information based on the correction information.

7. The method of claim 1, further comprising:
maintaining one or more variant call format (VCF) files based on the sample information.

8. The method of claim 1, wherein sending movement information to one or more robotics units comprises:
communicating with at least one vision system in order to identify a physical location of at least one sample.

9. The method of claim 1, further comprising:
communicating with at least one pneumatics system in order to control movement of at least one sample.

10. The method of claim 1, further comprising:
dynamically routing samples from a sample acquisition stage to a sequencing stage, wherein an automation process facilitates the dynamic routing based on stateful data and defined workflow processes.

11. The method of claim 1, further comprising:
retrieving stateful data and defined workflow processes;
generating one or more robotic move commands based on at least the stateful data and defined workflow processes;
communicating the one or more robotic move commands to the one or more robotics units in order to achieve high throughput sequencing.

12. A system for managing information in a laboratory, the system comprising:
a display;
one or more processors; and
a memory storing one or more programs, wherein the one or more programs include instructions configured to be executed by the one or more processors, causing the one or more processors to perform operations comprising:
receiving sample information, the sample information including at least one sample identifier and sample order information;
sending movement information to one or more robotics units based on at least the sample identifier;
performing, on at least one identified sample, one or more analytical functions to generate results data; and
organizing the results data based on the sample order information.

13. The system of claim 12, wherein the sample order information includes a disease panel order.

14. The system of claim 12, wherein instructions for organizing the results data further comprise instructions for:
rendering a graphical user interface to allow a user to manipulate the results data; and
storing updated results data based on one or more user inputs.

15. The system of claim 12, the one or more programs further comprising instructions for:
receiving a user command including correction information for a sample plate; and
storing positional information based on the correction information.

16. The system of claim 12, the one or more programs further comprising instructions for:
maintaining one or more variant call format (VCF) files based on the sample information.

17. The system of claim 12, wherein instructions for sending movement information to one or more robotics units further comprise instructions for:
communicating with at least one vision system in order to identify a physical location of at least one sample.

18. The system of claim 12, wherein instructions for sending movement information to one or more robotics units further comprise instructions for:
communicating with at least one pneumatics system in order to control the movement of at least one sample.

19. The system of claim 12, the one or more programs further comprising instructions for:
dynamically routing samples from a sample acquisition stage to a sequencing stage, wherein an automation process facilitates the dynamic routing based on stateful data and defined workflow processes.

20. The system of claim 12, the one or more programs further comprising instructions for:
retrieving stateful data and defined workflow processes;
generating one or more robotic move commands based on at least the stateful data and defined workflow processes;
communicating the one or more robotic move commands to the one or more robotics units in order to achieve high throughput sequencing.

21. The system of claim 12, wherein the sample order information includes an assay order.

22. The system of claim 12, wherein the sample order information includes an indication that the at least one identified sample is for research or for production.

23. A non-transitory computer readable storage medium having instructions stored thereon, the instructions, when executed by one or more processors, cause the processors to perform operations comprising:
receiving sample information, the sample information including at least one sample identifier and sample order information;
sending movement information to one or more robotics units based on at least the sample identifier;
performing, on at least one identified sample, one or more analytical functions to generate results data; and
organizing the results data based on the sample order information.

24. The storage medium of claim 23, wherein the sample order information includes a disease panel order.

25. The storage medium of claim 23, further comprising instructions for:
rendering a graphical user interface to allow a user to manipulate the results data; and
storing updated results data based on one or more user inputs.

26. The storage medium of claim 23, further comprising instructions for:
receiving a user command including correction information for a sample plate; and
storing positional information based on the correction information.

27. The storage medium of claim 23, further comprising instructions for:
maintaining one or more variant call format (VCF) files based on the sample information.

28. The storage medium of claim 23, wherein instructions for sending movement information to one or more robotics further comprise instructions for:
communicating with at least one vision system in order to identify a physical location of at least one sample.

29. The storage medium of claim 23, further comprising instructions for:
communicating with at least one pneumatics system in order to control the movement of at least one sample.

30. The storage medium of claim 23, further comprising instructions for:
dynamically routing samples from a sample acquisition stage to a sequencing stage, wherein an automation process facilitates the dynamic routing based on stateful data and defined workflow processes.

31. The storage medium of claim 23, further comprising instructions for:
retrieving stateful data and defined workflow processes;
generating one or more robotic move commands based on at least the stateful data and defined workflow processes;
communicating the one or more robotic move commands to the one or more robotics units in order to achieve high throughput sequencing.

32. The storage medium of claim 23, wherein the sample order information includes an assay order.

33. The storage medium of claim 23, wherein the sample order information includes an indication that the at least one identified sample is for research or for production.
